(19) United States
(12) Patent Application Publication
(10) Pub. No.: US 2020/0258611 A1
(43) Pub. Date: Aug. 13, 2020

(54) NOVEL MULTI-COMPARTMENT DOSAGE CARDS FOR SPLIT PACKAGE ADMINISTRATION

(71) Applicant: Bhuren Patel, Needham, MA (US)

(72) Inventor: Bhuren Patel, Needham, MA (US)

(21) Appl. No.: 16/863,019

(22) Filed: Apr. 30, 2020

Related U.S. Application Data
(63) Continuation of application No. 14/936,653, filed on Nov. 9, 2015.
(60) Provisional application No. 62/077,303, filed on Nov. 9, 2014.

(51) Int. Cl. G16H 20/10; A61J 1/03

(52) U.S. Cl. CPC G16H 20/10; A61J 1/03

(57) ABSTRACT

The present invention relates to novel technology suitable for reducing errors introduced into the process of medication administration. In particular, the present invention provides an instrument for on demand printing of patient database information directly to medication packaging, e.g., multi-compartment packaging, through a patient database interfacing control unit (DICU), which is capable of generating a dose label for a multi-compartment package, wherein each compartment label of the complete package label is independently compliant to state guidance documents incorporating the requirements of the five rights for split package administration. Accordingly, the present invention provides multi-compartment dosage cards engineered for safer split package administration, and systems to produce such cards.

### RELATED APPLICATIONS

This application claims priority to co-pending U.S. Utility application Ser. No. 14/936,653, filed Nov. 9, 2015, under Attorney Docket No. PCP-003, and which claims the benefit of priority of U.S. Provisional Patent Application No. 62/077,303, filed on Nov. 9, 2014, under Attorney Docket No. PCP-003-1; the entirety of each of which is incorporated herein by reference.

### BACKGROUND OF THE INVENTION

The process of medication administration has long been plagued with problems on both the patient and the prescriber sides. Whether the issue is patient compliance with prescribed medication, or the integrity of the process of prescription, ensuring that the right patient takes the right medication at the right time has become a tenet of the industry of medication administration. In fact, in order to specifically reduce errors in medication administration, the industry standard of care has moved towards general guidance known as the “five rights,” which focuses on the performance of individuals involved in the chain of medication administration.

Generally regarded as a standard for safe medication administration practices, the five rights are intended to guide the process of administration of (1) the right drug at (2) the right strength to (3) the right patient via (4) the right route at (5) the right time. In this way, it is believed that competent and safe care is reinforced by “the five rights”, which are aimed at ensuring the proper implementation and information communication. Although the focus of the five rights is on the performance of individuals, these individual efforts culminate in the entire process of delivering the drug to the patient in accordance with the doctor's instructions, which includes the efforts of a number of professionals from various disciplines; wherein the responsibility for accurate drug administration lies with multiple individuals and their part in reliable systems.

However, although these criteria mark the goals of safe medication practice, many errors, including lethal mistakes, have occurred even when health care professionals were confident that they had verified these “rights.” Clearly the number of steps that are involved in the process of medication administration creates, at each step, the ability to introduce errors. In this respect, and with increased risk, the “split package administration” of medication in group homes and organizations charged with the healthcare of others introduces an additional step into this process, whereby certified or licensed individuals may separate and administer portions of full multi-dosage prescriptions in accordance with state guidance documents, only by transferring information related to the five rights by hand to a new document or container loosely associated with the split package administered medication.

Accordingly, there is significant need for additional systems and packaging that address these concerns and reduce the number of potential errors that could be introduced, more sufficiently adhering to the five rights.

### SUMMARY OF THE INVENTION

Accordingly, the present invention is directed to novel technology suitable for reducing errors introduced into the process of medication administration. In particular, the present invention provides an instrument for on demand printing of patient database information directly to medication packaging, e.g., multi-compartment packaging, through a patient database interfacing control unit (DICU), which is capable of generating a dose label for a multi-compartment package, wherein each compartment label of the complete package label is independently compliant to state guidance documents incorporating the requirements of the five rights for split package administration. Accordingly, the present invention provides multi-compartment dosage cards engineered for safer split package administration, and systems to produce such cards.

As such, one aspect of the invention provides a multi-compartment dosage card, engineered for split package administration. The multi-compartment dosage card comprises two or more sealed medication compartments comprising connected and fillable medication containment wells sealed with a containment sealing material that is capable of serving as a receiving surface for direct printing, wherein each sealed medication compartment is capable of being separated from the other medication compartments while retaining the integrity of each sealed medication compartment. The multi-compartment dosage card also comprises a complete dose label for a multi-compartment dosage card generated from a compliance data bundle selected from data in a patient database, wherein each printed compartment label for each medication compartment is independently compliant for split package administration, and wherein said complete dose label is printed directly on the containment sealing material.

Another aspect of the present invention provides a patient database interfacing control unit (DICU) suitable for unifying patient database information with package printing technology comprising a machine-readable medium having instructions stored thereon for execution by a processor. The processor performs a method comprising the steps of: interfacing with a patient database to access data entered into the patient database; accessing the data entered into the patient database; selecting a compliance data bundle from the data in the patient database; generating a complete dose label for a multi-compartment dosage card from the compliance data bundle, wherein each compartment label of the complete dose label is independently compliant for split package administration; and printing the complete dose label on said multi-compartment dosage card, e.g., a unit dose, e.g., a blister card.

In another aspect, the present invention provides a system, e.g., a turn key system, for producing a multi-compartment dosage card, engineered for split package administration. The system comprises a medication filling unit that deposits at least one medication into two or more connected and fillable medication containment wells; a sealing apparatus that seals the filled medication containment wells with a containment sealing material that is capable of serving as a receiving surface for direct printing to form sealed medication compartments, wherein each sealed medication compartment is capable of being separated from the other medication compartments while retaining the integrity of each sealed medication compartment; a patient database; a patient database interfacing control unit (DICU) suitable for unifying patient database information with package printing technology comprising a machine-readable medium having instructions stored thereon for execution by a processor to perform a method comprising the steps of:
interfacing with a patient database to access data entered into the patient database;
accessing the data entered into the patient database;
selecting a compliance data bundle from the data in the patient database;
generating a complete dose label for a multi-compartment dosage card from the compliance data bundle, wherein each compartment label of the complete dose label is independently compliant for split package administration; and
printing the complete dose label on said multi-compartment dosage card, e.g., a unit dose, e.g., a blister card.
   The system also comprises a printing device that prints the complete dose label on said multi-compartment dosage card, such that a multi-compartment dosage card engineered for split package administration is produced.

In yet another aspect, the present invention provides a multi-compartment dosage card produced by the system for producing a multi-compartment dosage cards described herein.

### BRIEF DESCRIPTION OF THE DRAWINGS

Advantages of the present apparatus will be apparent from the following detailed description, which description should be considered in combination with the accompanying drawings, which are not intended limit the scope of the invention in any way.

FIG. 1 depicts an image with top down perspective view of certain particular embodiments of the multi-compartment dosage cards of the present invention.

FIG. 2 depicts an image with a bottom up perspective view of certain particular embodiments of the multi-compartment dosage cards of the present invention, i.e., the multi-compartment dosage card of FIG. 1.

FIG. 3 depicts an image with top down perspective view of certain particular embodiments of the multi-compartment dosage cards of the present invention, e.g., of FIG. 1, further showing the separation of a compartment for split package administration.

FIG. 4 depicts an image with a bottom up perspective view of certain particular embodiments of the multi-compartment dosage cards of the present invention, i.e., the multi-compartment dosage card of FIG. 3.

### DETAILED DESCRIPTION OF THE INVENTION

The present invention is directed to novel technology suitable for reducing errors introduced into the process of medication administration. In particular, the present invention provides a mechanism for on-demand printing of patient database information directly to medication packaging, e.g., multi-compartment packaging, through a patient database interfacing control unit (DICU), which is capable of generating a dose label for a multi-compartment package, wherein each compartment label of the complete package label is independently compliant to state guidance documents incorporating the requirements of the five rights for split package administration.

Packaging in compliance with medication administration programs have generally satisfied labeling requirements by using a single primary label with the required statutory information, and provided limited handwritten information relating to the five rights for split package administration, e.g., for a leave of absence (LOA), which is transferred from the primary label by a certified or licensed individual. In particular, during split package administration, one or more doses are separated from the primary label, and therefore the patient to whom the medication is administered receives a portable but vulnerable, e.g., error susceptible, form of the five rights through this handwritten transference of information; which not only introduces an additional step, but one that is predisposed for transcription errors.

The present invention provides a safer, and more convenient solution to the problems related to split packaging of medication dosages through the discovery of a mechanisms described herein for printing certain data related to the five rights, e.g., from a compliance bundle, on each compartment of a multi-compartment dosage card. As such, the data on the printed label is directly derived from a patient database without interruption, and without the potential introduction of errors. The elimination of this transference step has wide market appeal, especially from those in the industry of state regulated healthcare, which must strictly adhere to the state guidelines for split package administration. Moreover, replacement of this step with the highly systematic process described herein, produced from the direct interface of the printing technology with the patient database, increases the safety for the patients not only by eliminating this transference step, but also reducing the overall number of vulnerable steps between prescription and administration (e.g., the step of separately printing out labels by an operator which are then affixed to the medication packaging).

In fact, the present invention exceeds current standards for state medication administration programs. As such, in certain embodiment, non-licensed or certified employees are afforded the ability to administer split package medications.

The present invention, including DICUs, multi-compartment packaging, and systems will be described with reference to the following definitions that, for convenience, are set forth below. Unless otherwise specified, the below terms used herein are defined as follows:

### 1. Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “automatically” is used herein to describe a process that is automated or semi-automated. Automated processes do not contain steps that require a human operator to perform the steps. Semi-automated processes contain one or more steps that require a human operator to perform the steps; however, differ from manual processes by containing at least one step that does not require a human operator.

The term “interfacing” as used herein, for example in the expression “interfacing with a patient database,” describes the means of communication between two entities, for example the patient database and the DICU. In certain embodiments, the interfacing may be bi-directional. In other embodiments, the interfacing may be uni-directional. In particular embodiments, such interfacing may include receiving, responding, and/or assigning an answer to a request, e.g., wherein the request is for a compliance data bundle from the patient database, e.g.; after access and/or selection of the data in the patient database.

The language “machine-readable medium” is art-recognized, and describes a medium capable of storing data in a format readable by a mechanical device (rather than by a human). Examples of machine-readable media include magnetic media such as magnetic disks, cards, tapes, and drums, punched cards and paper tapes, optical disks, barcodes, magnetic ink characters, and solid state devices such as flash-based, SSD, etc. Machine-readable medium of the present invention are non-transitory, and therefore do not include signals per se, i.e.; are directed only to hardware storage medium. Common machine-readable technologies include magnetic recording, processing waveforms, and barcodes. In particular embodiments, the machine-readable device is a solid state device. Optical character recognition (OCR) can be used to enable machines to read information available to humans. Any information retrievable by any form of energy can be machine-readable. Moreover, any data stored on a machine-readable medium may be transferred by streaming over a network.

The term “medication” is art-recognized, and is used herein to describe a drug in a form suitable for administration by the packages of the present invention, e.g., as a pill.

The language “patient database” is art-recognized, and is used herein to describe a database of patient and patient-specific prescription information. For example, in one embodiment, the patient database is pharmacy database software, e.g., Suite Rx, QS1, or Rx30.

The language “turn-key” as used herein describes a system comprised of processes that are automated to the extent that a single input produces a final output without any additional manual steps.

### II. Multi-Compartment Dosage Cards of the Invention

Accordingly, the present invention provides multi-compartment packaging engineered for safer split package administration, and systems to produce such packaging. In particular, the present invention provides multi-compartment dosage cards, e.g., tamper evident/resistant, which increase the safety for the patients not only by eliminating an error susceptible transference step, but also reducing the overall number of vulnerable steps between prescription and administration (e.g., the step of separately printing out labels by an operator which are then affixed to the medication packaging).

One embodiment of the invention provides a multi-compartment dosage card, engineered for split package administration comprising
two or more sealed medication compartments comprising connected and fillable medication containment wells sealed with a containment sealing material that is capable of serving as a receiving surface for direct printing, wherein each sealed medication compartment is capable of being separated from the other medication compartments while retaining the integrity of each sealed medication compartment; and
a complete dose label for a multi-compartment dosage card generated from a compliance data bundle selected from data in a patient database, wherein each printed compartment label for each medication compartment is independently compliant for split package administration, and wherein said complete dose label is printed directly on the containment sealing material.

In certain embodiments, the multi-compartment dosage card, e.g., blister pack, comprises an affixed primary label that satisfies the requisite prescription requirements of the Federal Food Drug & Cosmetic Act, for example, indicating one or more of the following:
i. the name of the individual;
ii. the name of the controlled substance (medication) including the IC (i.e., interchange) name, if any, e.g., the IC name of the medication may be listed when the pharmacy dispenses a generic substitution or a less expensive brand name drug product for the prescribed medication;
iii. the strength and amount;
iv, the frequency;
v. the route of administration;
vi. the name of the prescribing health care provider;
vii. directions for use;
viii. cautionary statements, if any;
ix. total quantity of medication dispensed (e.g., if the medication was dispensed as tablets or capsules, the number of same in the container);
x. the date of filling;
xi. the lot number;
xii. the pharmacy name, address, telephone number and serial/prescription number;
xiii. the expiration date; and
iv. the filling pharmacist's initials.

The multi-compartment dosage cards of the present invention are designed for split package administration, and therefore afford the user the ability to offer one or more doses that are separated from the primary label (i.e., the patient receives only a portion of the originally dispensed medication). These separated doses retain a label which is printed directly on the sealing material, e.g., a compartment label, comprising patient specific information that complies with the five rights; where, in contrast to existing protocol, this information is not transferred from the primary label by a certified or licensed individual, but rather is directly obtained from the same source as the primary label, i.e., the patient database.

The multi-compartment dosage cards of the present invention are packaging in compliance with medication administration programs (MAP), e.g., compliant with the Massachusetts Medication Administration Program policies.

In particular embodiments of the present invention, the patient specific information on the printed compartment label is selected from
1. individual's name;
2. name and strength of medication;
3. directions for usage (e.g., clearly stated; including specific doses, dosing times, and dosing route);
4. prescribing health care provider's name;
5. date of dispensing;
6. any necessary cautionary statements (e.g., take with food.); and
7. amount of medication,
   or any combination thereof. In a specific embodiment of the present invention, the patient specific information on the printed compartment label is selected from
1. individual's name;
2. name and strength of medication;
3. directions for usage (e.g., clearly stated; including specific doses, dosing times, and dosing route); and
4. amount of medication.

In certain embodiments of the present invention, the multi-compartment dosage cards of the present invention are tamper-resistant (i.e., packaged in such a manner that prevents the contents from being altered). In certain embodiments of the present invention, the multi-compartment dosage cards of the present invention are tamper-evident (i.e., packaged in such a manner that allows relatively easy detection by a user if the contents have been altered).

In certain embodiments of the invention, the multi-compartment dosage card is a multi-compartment dosage card produced by the systems of the present invention.

A. Containment Wells/Sealed Medication Compartments

The multi-compartment dosage cards of the present invention comprise containment wells, which are connected or linked in some manner, and which are designed to be filled by medication. Once sealed, e.g., after filling with medication, with a sealing material, the containment wells form a sealed medication compartment. As such, the sealed medication compartments will also be linked upon sealing. The linkage or connection may be a direct connection, e.g., being part of the same casting, or may be connected indirectly for example the wells sit in another array, wherein the array provides the linking. In a particular embodiment, the wells are connected by being part of the same thermoformed plastic array casting comprising cavities that serve as the wells to hold the medication.

Moreover, the containment wells of the multi-compartment dosage cards of the present invention are connected or joined together in such a manner as to be suitable for separation after sealing by the sealing material, e.g., by size, spacing, and positioning in the compartment array. Upon separation from the other medication compartments, a single compartment retains the integrity of the sealed medication compartment, i.e., remains unadulterated and not misbranded (e.g., untampered).

In certain embodiments of the invention, the multi-compartment dosage card of the invention further comprises a single medication in each medication compartment.

In certain embodiments of the invention, the multi-compartment dosage card of the invention further comprises multiple medications in each medication compartment.

In certain embodiments of the invention, the medication is a single dosage.

In certain embodiments of the invention, the fillable medication containment well is manually fillable with medication. In particular, the medication containment well is engineered to be filled with medication through manual filling.

In certain embodiments of the invention, the fillable medication containment well is automatically fillable with medication, e.g., able to deliver medications of various shapes and sizes. In particular, the medication containment well is engineered to be filled with medication automatically, e.g., by a medication filling unit.

In certain embodiments of the invention, the materials comprising the multi-compartment dosage card may be selected from the group consisting of any material allowed to be used according to the Food Drug & Cosmetic Act, e.g., according to FDA guidelines, e.g., any one or more of the materials selected from the FDA list of food contact substances (FCS), including, for example, foil, plastic (e.g., thermoformed plastic) and any combination thereof.

In certain embodiments of the invention, the materials comprising the multi-compartment dosage card may be selected from the group consisting of foil, plastic (e.g., thermoformed plastic) and any combination thereof.

In certain embodiments of the invention, at least one of the materials is a structured material capable of receiving an insert comprising said connected and fillable medication containment wells.

The sealing material may be any material suitable for sealing the containment wells and serving as a receiving surface for direct printing by a printer. In certain embodiments of the invention, the containment sealing material is comprised of one or more materials.

In certain embodiments of the invention, the containment sealing material is comprised of foil and card stock. In particular embodiments, the fillable medication containment wells, e.g., made of thermoformed plastic, sit inside the card stock that is designed for receiving the wells, and the foil is positioned to seal the fillable medication containment wells sitting in the card stock. In a particular embodiment, the multi-compartment dosage card further comprises a backing card stock identically shaped to the card stock used for receiving the fillable medication containment wells, and positioned to seal the foil in between the two card stock layers.

In certain embodiments of the invention, the fillable medication containment wells sealed with a containment sealing material is sealed using a heated processing step, e.g., a standard heat seal.

In certain embodiments of the invention, the fillable medication containment wells sealed with a containment sealing material is sealed using a cold processing step.

In certain embodiments of the invention, the sealed medication compartments form a blister pack orientation.

In certain embodiments of the invention, the sealed medication compartments are connected by perforations, which upon separation from the multi-compartment dosage through tearing of the perforation can produce an independently compliant single compartment for split package administration.

In certain embodiments of the invention, the sealed medication compartments are not connected by perforations, which upon separation from the multi-compartment dosage through cutting or tearing produce an independently compliant single compartment for split package administration.

In certain embodiments of the invention, the multi-compartment dosage card meets Class B Container requirements, e.g., Class B container assures a dependable barrier against moisture and gasses.

In certain embodiments of the invention, the number of sealed medication compartments is selected from the group consisting of 30, 60, and 90.

In certain embodiments of the invention, the multi-compartment dosage card is color-coded, e.g., for added clarity on timing of administration.

In certain embodiments of the invention, the multi-compartment dosage card is bar coded for medication verification and tracking, e.g., on the front and/or back of the card.

B. Dose Labels

The complete dose label for the multi-compartment dosage cards of the present invention is printed directly on the containment sealing material, and is comprised of two or more compartment labels designed to be positioned cleanly in a manner that affords the ability to remove one compartment from the others while retaining the entirety of the compartment label with the sealed medication compartment. The complete dose label is generated from a compliance data bundle selected from data in a patient database. This compliance data bundle is the electronic data packet comprising patient data selected from a patient database which is equivalent to the five rights data, e.g., that which would be transferred by hand by a licensed/certified staff member of a medical administration program.

As such, each printed compartment label for each medication compartment is independently compliant for split package administration, comprising patient specific information that complies with the five rights; where, in contrast to existing/known protocols, this information is not transferred from the primary label by a certified or licensed individual, but rather is directly obtained from the same source as the primary label, i.e., the patient database. Doses separated from the primary label for split package administration retain a label which is printed directly on the sealing material, e.g., a compartment label, which affords the user the ability to administer one or more doses that are each compliant for split package administration, e.g., according to medication administration programs, e.g., compliant with the Massachusetts Medication Administration Program policies

In certain embodiments, the packaging of the present invention is obtained through the use a patient database interfacing control unit.

### III. Patient Database Interfacing Control Unit (DICU) of the Invention

Another embodiment of the present invention provides a patient database interfacing control unit (DICU) suitable for unifying patient database information with package printing technology comprising a machine-readable medium having instructions stored thereon for execution by a processor to perform a method comprising the steps of:

interfacing with a patient database to access data entered into the patient database;

accessing the data entered into the patient database;

selecting a compliance data bundle from the data in the patient database;

generating a complete dose label for a multi-compartment dosage card from the compliance data bundle, wherein each compartment label of the complete dose label is independently compliant for split package administration; and

printing the complete dose label on said multi-compartment dosage card, e.g., a unit dose, e.g., a blister card. In particular, the DICU is software that enables on-demand printing of patient information from a patient database, wherein the selected patient information sufficient to satisfy the five rights of patient safety is printed on one or more compartments, i.e., the compartment label(s). Moreover, it affords the ability to search through the database, select certain information for printing, and then transfer such information to a printer, e.g., a printer driver, for printing on the multi-compartment dosage card.

In certain embodiments of the patient database interfacing control unit (DICU), the multi-compartment dosage card is a multi-compartment dosage card of the invention.

### IV. Systems of the Invention

Another embodiment of the present invention provides a system, e.g., a turn key system, for producing a multi-compartment dosage card, engineered for split package administration comprising:
a medication filling unit that deposits at least one medication into two or more connected and fillable medication containment wells;
a sealing apparatus that seals the filled medication containment wells with a containment sealing material that is capable of serving as a receiving surface for direct printing to form sealed medication compartments, wherein each sealed medication compartment is capable of being separated from the other medication compartments while retaining the integrity of each sealed medication compartment;
a patient database, e.g., Suite Rx, QS1, or Rx30;
a patient database interfacing control unit (DICU) suitable, e.g., as described herein, for unifying patient database information with package printing technology comprising a machine-readable medium having instructions stored thereon for execution by a processor to perform a method comprising the steps of:
   interfacing with a patient database to access data entered into the patient database;
   accessing the data entered into the patient database;
   selecting a compliance data bundle from the data in the patient database;
   generating a complete dose label for a multi-compartment dosage card from the compliance data bundle, wherein each compartment label of the complete dose label is independently compliant for split package administration; and
   printing the complete dose label on said multi-compartment dosage card, e.g., a unit dose, e.g., a blister card; and
a printing device that prints the complete dose label on said multi-compartment dosage card,
   such that a multi-compartment dosage card engineered for split package administration is produced.

In certain embodiments of the invention, said multi-compartment dosage card is a multi-compartment dosage card of the invention, as described herein.

### A. Medication Filling Unit

Systems of the present invention comprise a medication filling unit that deposits at least one medication into two or more connected and fillable medication containment wells. In certain embodiments, the medication filling unit deposits one medication. The medication filling unit may operate automatically (e.g., automated or semi-automated) or manually. In certain embodiments, filling with medication may be performed with package filling systems known in the art.

The medication filling unit is used to fill the containment wells that may be designed and or arranged to receive and retain medication, e.g., individual doses of medication. In certain embodiments, the containment wells are arranged into an array of individual cavities suitable for receiving and retaining the medication. In particular embodiments, one or more transmission channels are provided, e.g., in one-to-one correspondence with the individual cavities of the wells, to transmit the medication from the medication filling unit to the containment well. Noting that when filling the containment wells with small pills, care may be taken to ensure that the transmission channel for the small pills is properly aligned with the cavity so that the small pills are properly transmitted to the appropriate package cavity location

### B. Sealing Apparatus

Systems of the present invention comprise a sealing apparatus that seals the filled medication containment wells with a containment sealing material that is capable of serving as a receiving surface for direct printing to form sealed medication compartments, wherein each sealed medication compartment is capable of being separated from the other medication compartments while retaining the integrity of each sealed medication compartment. In certain embodiments, such sealing system may be any sealing system known in the art adapted for the use in the present invention, e.g., utilizing a heat seal or a cold seal processing step.

### C. Printing Device

Systems of the present invention comprise a printing device that prints the complete dose label on said multi-compartment dosage card. Such printing device may be any printer suitable for printing on the containment sealing material alone or as part of a sealed package in a manner that affords readability to the compartment labels comprising data from the compliance data bundle.

In a particular embodiment, the printing device comprises a printer with a straight through paper feed. In specific embodiments, the printing device comprises a printer designed to print on heavier cardstock, e.g., an inkjet printer (e.g., an Epson artisan 1430 inkjet printer) or an LED printer (e.g., an OKI Data LED printer, e.g., model C931D9).

### D. Additional Embodiments

In certain embodiments of the invention, the system further comprises said connected and fillable medication containment wells.

In certain embodiments of the invention, the system further comprises said containment sealing material.

In certain embodiments of the invention, the system further comprises a single medication in each medication compartment. In particular embodiments, the medication is a single dosage.

In certain embodiments of the invention, the system further comprises multiple medications in each medication compartment. In particular embodiments, the medication is a single dosage.

### V. Design Aspects of the Invention

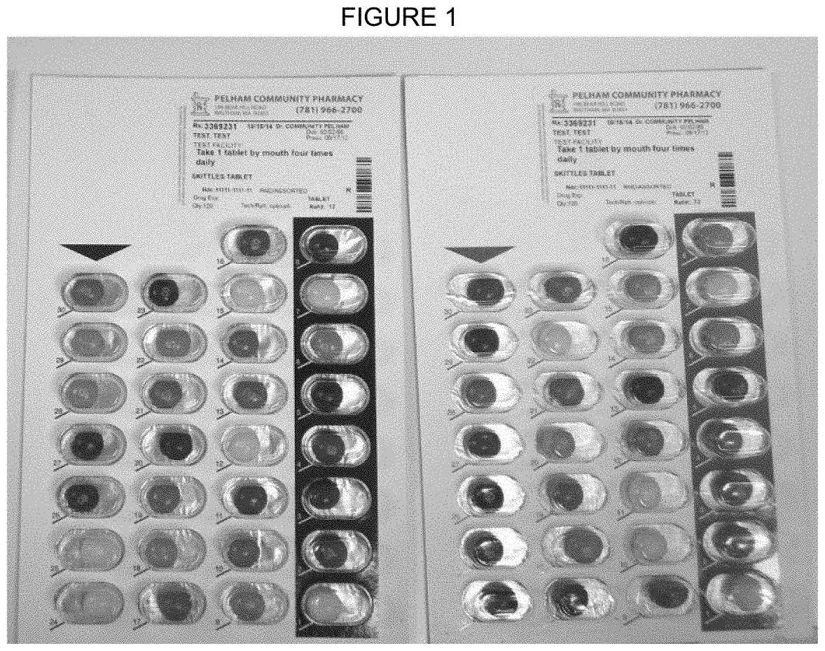

Independent of the utility related to multi-compartment dosage cards of the present invention, the ornamental appearance of any novel design provided herein is intended to be part of this invention, for example, each of the perspective views in FIGS. 1 through 4, which may form an independent or combined ornamental appearance of multi-compartment dosage cards described herein.

Accordingly, one embodiment of the present invention provide an ornamental design for a multi-compartment dosage card as shown and described.

### EXEMPLIFICATION

Having thus described the invention in general terms, reference will now be made to the accompanying drawings of exemplary embodiments, which are not necessarily drawn to scale, and which are not intended to be limiting in any way.

In this respect, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

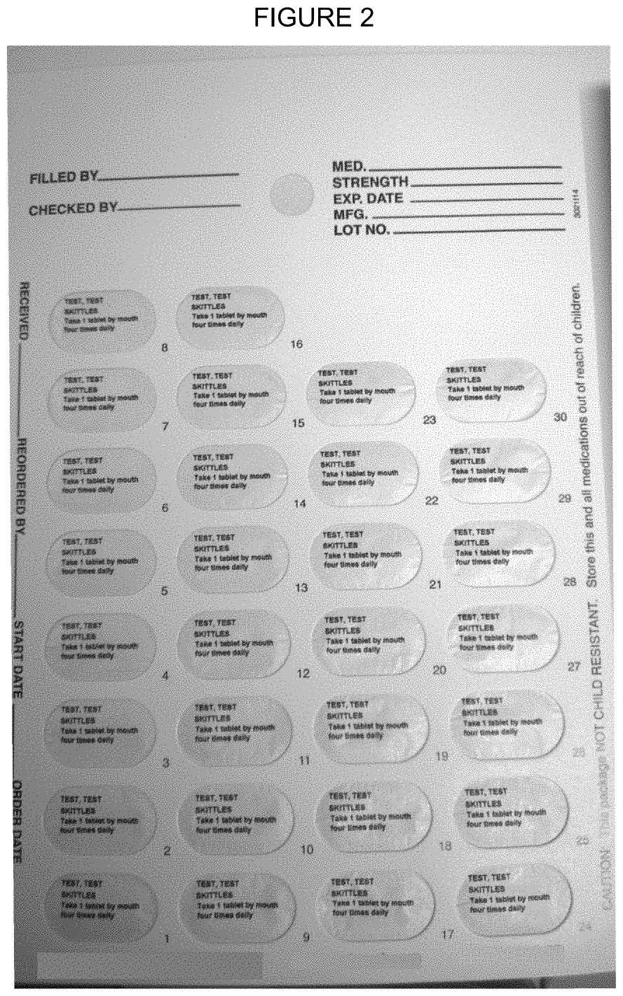

FIG. 1, a top down perspective view of certain particular embodiments of the multi-compartment dosage cards of the present invention, depicts 30 sealed medication compartments filled with 30 unit dosages of a medication, i.e., one in each compartment. The fillable medication containment wells are made of a thermoformed plastic, casted in a particular array of fillable wells, which are connected through this thermoformed plastic. The array of containment wells are sealed by a foil containment sealing material, which is sealed in between two card stock layers, wherein the visible front layer is structured card stock material capable of receiving an insert of the thermoformed plastic containment wells. The second card stock layer is visible in FIG. 2, a bottom up perspective view of the multi-compartment dosage card of FIG. 1, which is backing card stock identically shaped to the card stock used for receiving the fillable medication containment wells, and positioned to seal the foil in between the two card stock layers

FIG. 2 depicts the back side of the foil containment sealing material which is capable of serving as a receiving surface for direct printing, and contains a complete dose label generated from a compliance data bundle selected from data in a patient database, e.g., of a test patient. Each printed compartment label for each medication compartment comprises a listing of information that is independently compliant for split package administration.

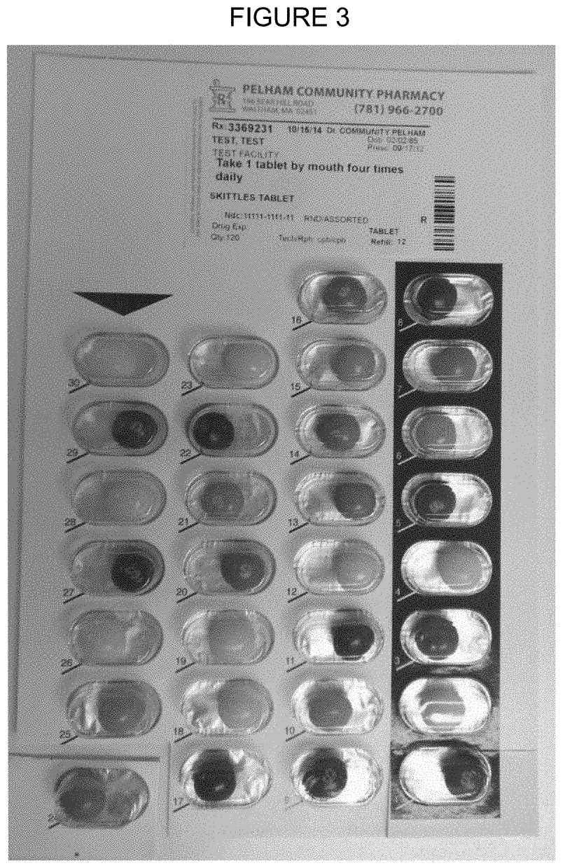

FIG. 3, a top down perspective view of certain particular embodiments of the multi-compartment dosage cards of the present invention, e.g., of FIG. 1, shows a single dosage portion in a sealed medication compartment separated from the other medication compartments, e.g., via cutting, which retains its integrity for split package administration. In addition, the remainder of the dosage portions in the other sealed medication compartments retain their integrity.

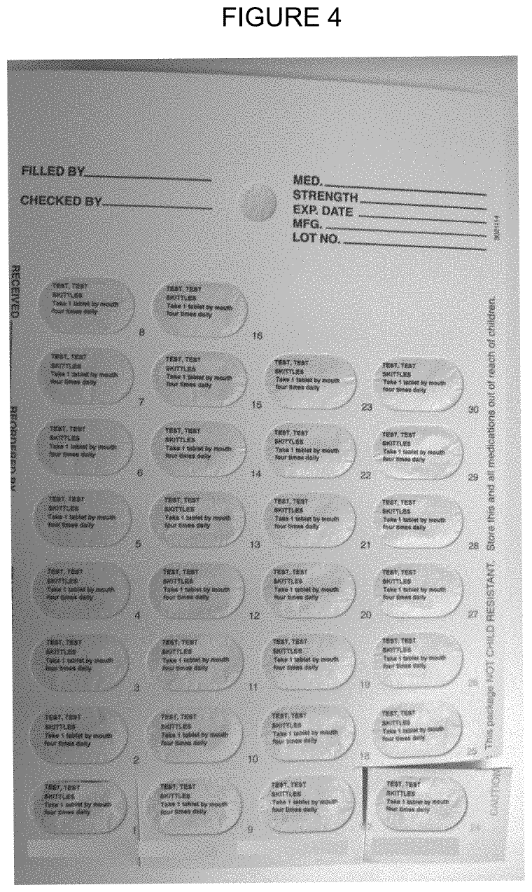

FIG. 4, a bottom up perspective view of certain particular embodiments of the multi-compartment dosage cards of the present invention, i.e., the multi-compartment dosage card of FIG. 3, confirms that the separated sealed medication compartment label is retained and compliant for split package administration.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

What is claimed is:

1. A multi-compartment dosage card, engineered for split package administration comprising
two or more sealed medication compartments comprising connected and fillable medication containment wells sealed with a containment sealing material that is capable of serving as a receiving surface for direct printing, wherein each sealed medication compartment is capable of being separated from the other medication compartments while retaining the integrity of each sealed medication compartment; and
a complete dose label for a multi-compartment dosage card generated from a compliance data bundle selected from data in a patient database, wherein each printed compartment label for each medication compartment is independently compliant for split package administration, comprising
the patient name; name and strength of medication; directions for usage, including specific doses, dosing times, and dosing route; and amount of medication, and
wherein said complete dose label is printed directly on the containment sealing material.

2. The multi-compartment dosage card of claim 1 further comprising a single medication in each medication compartment.

3. The multi-compartment dosage card of claim 1 further comprising multiple medications in each medication compartment.

4. The multi-compartment dosage card of claim 1, wherein the medication is a single dosage.

5. The multi-compartment dosage card of claim 1, wherein the fillable medication containment well is manually fillable with medication.

6. The multi-compartment dosage card of claim 1, wherein the fillable medication containment well is automatically fillable with medication.

7. The multi-compartment dosage card of claim 1, wherein the containment sealing material is comprised of one or more materials.

8. The multi-compartment dosage card of claim 1, wherein the multi-compartment dosage card comprises materials selected from the group consisting of foil, plastic, card stock, and any combination thereof.

9. The multi-compartment dosage card of claim 7, wherein at least one of the materials is a structured material capable of receiving an insert comprising said connected and fillable medication containment wells.

10. The multi-compartment dosage card of claim 1, wherein the containment sealing material is comprised of foil and card stock.

11. The multi-compartment dosage card of claim 10, wherein the fillable medication containment wells sit inside the card stock that is designed for receiving the wells, and the foil is positioned to seal the fillable medication containment wells sitting in the card stock.

12. The multi-compartment dosage card of claim 11 further comprising a backing card stock identically shaped to the card stock used for receiving the fillable medication containment wells, and positioned to seal the foil in between the backing card stock and the card stock used for receiving the fillable medication containment wells.

13. The multi-compartment dosage card of claim 1, wherein the fillable medication containment wells sealed with a containment sealing material is sealed using a heated processing step.

14. The multi-compartment dosage card of claim 1, wherein the fillable medication containment wells sealed with a containment sealing material is sealed using a cold processing step.

15. The multi-compartment dosage card of claim 1, wherein the sealed medication compartments form a blister pack orientation.

16. The multi-compartment dosage card of claim 1, wherein the sealed medication compartments are connected by perforations, which upon separation from the multi-compartment dosage through tearing of the perforation can produce an independently compliant single compartment for split package administration.

17. The multi-compartment dosage card of claim 1, wherein the multi-compartment dosage card meets Class B Container requirements.

18. The multi-compartment dosage card of claim 1, wherein the number of sealed medication compartments is selected from the group consisting of 30, 60, and 90.

19. The multi-compartment dosage card of claim 1, wherein the multi-compartment dosage card is color-coded.

20. The multi-compartment dosage card of claim 1, wherein the multi-compartment dosage card is bar coded for medication verification and tracking.

21. A patient database interfacing control unit (DICU) suitable for unifying patient database information with package printing technology comprising a machine-readable medium having instructions stored thereon for execution by a processor to perform a method comprising the steps of:
interfacing with a patient database to access data entered into the patient database;
accessing the data entered into the patient database;
selecting a compliance data bundle from the data in the patient database;
generating a complete dose label for a multi-compartment dosage card from the compliance data bundle, wherein each compartment label of the complete dose label is independently compliant for split package administration, comprising
the patient name; name and strength of medication; directions for usage, including specific doses, dosing times, and dosing route; and amount of medication; and
printing the complete dose label on said multi-compartment dosage card.

22. The patient database interfacing control unit (DICU) of claim 21, wherein the multi-compartment dosage card is a multi-compartment dosage card engineered for split package administration comprising
two or more sealed medication compartments comprising connected and fillable medication containment wells sealed with a containment sealing material that is capable of serving as a receiving surface for direct printing, wherein each sealed medication compartment is capable of being separated from the other medication compartments while retaining the integrity of each sealed medication compartment; and
a complete dose label for a multi-compartment dosage card generated from a compliance data bundle selected from data in a patient database, wherein each printed compartment label for each medication compartment is independently compliant for split package administration, and wherein said complete dose label is printed directly on the containment sealing material.

23. A system for producing a multi-compartment dosage card, engineered for split package administration comprising:
a medication filling unit that deposits at least one medication into two or more connected and fillable medication containment wells;
a sealing apparatus that seals the filled medication containment wells with a containment sealing material that is capable of serving as a receiving surface for direct printing to form sealed medication compartments, wherein each sealed medication compartment is capable of being separated from the other medication compartments while retaining the integrity of each sealed medication compartment;
a patient database;
a patient database interfacing control unit (DICU) suitable for unifying patient database information with package printing technology comprising a machine-readable medium having instructions stored thereon for execution by a processor to perform a method comprising the steps of:
interfacing with a patient database to access data entered into the patient database;
accessing the data entered into the patient database;
selecting a compliance data bundle from the data in the patient database;
generating a complete dose label for a multi-compartment dosage card from the compliance data bundle, wherein each compartment label of the complete dose label is independently compliant for split package administration, comprising
the patient name; name and strength of medication; directions for usage, including specific doses, dosing times, and dosing route; and amount of medication; and
printing the complete dose label on said multi-compartment dosage card; and
a printing device that prints the complete dose label on said multi-compartment dosage card,
such that a multi-compartment dosage card engineered for split package administration is produced.

24. The system of claim 23, further comprising said connected and fillable medication containment wells.

25. The system of claim 23, further comprising said containment sealing material.
